(19) United States
(12) Patent Application Publication
(10) Pub. No.: US 2025/0041290 A1
(43) Pub. Date: Feb. 6, 2025

(54) USE OF N-(3-(4-(3-(DIISOBUTYLAMINO)PROPYL)PIPERAZIN-1-YL)PROPYL)-1H-BENZO[d]IMIDAZOL-2-AMINE SUCCINATE SALTS AND SOLVATES THEREOF FOR THE TREATMENT OF MOTOR NEURON DISEASES AND NEUROMUSCULAR JUNCTION DISORDERS

(71) Applicant: ALZPROTECT, Loos (FR)

(72) Inventors: Noëlle CALLIZOT, Aix-en-Provence (FR); Cécilia ESTRELLA, Lille (FR); Philippe VERWAERDE, Santes (FR); Cyrille BRANTIS, Marcq-en Baroeul (FR); Stéphane BURLET, Croix (FR)

(21) Appl. No.: 18/717,162

(22) PCT Filed: Dec. 19, 2022
(86) PCT No.: PCT/EP2022/086773

§ 371 Date: Jun. 6, 2024

(30) Foreign Application Priority Data
Dec. 20, 2021 (EP) 21306863.8

(51) Int. Cl. A61K 31/496

(52) U.S. Cl. CPC A61K 31/496

(57) ABSTRACT

The present invention relates to succinate salts of N-(3-(4-(3-(diisobutylamino)propyl)piperazin-1-yl)propyl)-1H-benzo[t/]imidazol-2-amine and pharmaceutically acceptable solvates thereof, for use in the treatment and/or prevention of motor neuron diseases and neuromuscular junction disorders.

The present invention relates to novel uses of N-(3-(4-(3-(diisobutylamino)propyl)piperazin-1-yl)propyl)-1H-benzo[d]imidazol-2-amine succinate salts and pharmaceutically acceptable solvates thereof, in the treatment and/or prevention of motor neuron diseases and neuromuscular junction disorders.

### BACKGROUND OF THE INVENTION

Motor neuron diseases (MND) and neuromuscular junction disorders (NJD) are both considered neuromuscular disorders which lead to muscle weakness and weariness that becomes more pronounced over time.

Motor neurons are neuronal cells present in the central nervous system (motor cortex, brainstem, spinal cord . . . ) and in the peripheral nervous system responsible for controlling organs like muscles and glands. Motor neurons are classified as upper or lower motor neurons. Upper motor neurons (UMN) are located in the cerebral cortex and in the brainstem, they transmit signals to interneurons and to lower motor neurons (LMN) via glutamatergic neurotransmission. The LMN are located in the spinal cord and innervate skeletal muscle fibers (neuromuscular junctions) where acetylcholine is released to carry the signal across the muscle cell membrane, signaling the muscle to contract or relax. Both UMN and LMN are essential to voluntary movements, balance, body posture adjustment and muscle control in general.

Progressive motor neuron degeneration is the hallmark of motor neuron diseases, it eventually causes motor neuron apoptosis and fasciculation of the motor units (unit defining the motor neuron—its axon—the neuromuscular junction—the individual muscle fibers it innervates altogether), preventing transmission of nerve influx controlling muscles. Death of the cell bodies of motoneurons is the primary process in MND and it occurs in conjunction with deposition of aggregated proteins in motoneurons and oligodendrocytes, as well as neuroinflammation. These diseases which can either affect UMN or LMN and even both, cause muscle weakness, hypotonia, movement lowness, impairment of reflexes and muscle atrophy.

There are numerous motor neuron diseases and amyotrophic lateral sclerosis (ALS) is the most common acquired motor neuron disease that could affect UMN and LMN. Sporadic ALS is the most common form of ALS (≥90% of cases) diagnosed in patients with no known family members affected by the disease. Familial ALS are of genetic origin and run in families, several gene mutations have been identified and linked thereto. The most common is in the chromosome 9 open reading frame 72 (C9ORF72) and can cause patients to suffer from cognitive defects as well: ALS-FTD is a particular form of ALS where patients also suffer from frontotemporal dementia (FTD). Amyotrophic lateral sclerosis with parkinsonism-dementia complex 1 (ALS-PDC) or Lytico-bodig disease, is a form of ALS where patients experience symptoms of ALS, dementia, and Parkinson's disease (PD). Other known genes associated with familial and sporadic ALS include those encoding superoxide dismutase I (SOD1), TAR DNA binding protein of 43-kDa (TDP-43), RNA-binding protein (FUS/TLS: Fused in Sarcoma/Translocated in Sarcoma) and ubiquilin 2 (UBQLN2). These mutations can cause toxic accumulation of these proteins in the motor neurons and astrocytes, thus subsequently leading to neurons death. TAR DNA binding protein of 43-kDa (TDP-43) is shown to accumulate in the cytoplasm of motor neurons in most cases of ALS. TDP-43 is a nuclear RNA-binding protein involved in several aspects of RNA processing that actively shuttles between the nucleus and the cytoplasm. In ALS, TDP-43 is excluded from the nucleus, but such cytoplasmic mislocalization is common in neuronal injury or stress, and TDP-43-positive inclusions may represent a secondary pathology in motor neuron diseases.

Other motor neuron diseases can either affect the UMN only such as primary lateral sclerosis (PLS) or the LMN only such as progressive muscular atrophy (PMA). Moreover, LMN degeneration and ALS can affect the neuromuscular junction (Dupuis L et al Curr Drug Targets 2010; 11 (10): 1250-1261-Gromova A et al, Trends Neurosci. 2020 September; 43 (9): 709-724—Hashizume A et al, J Neurol Neurosurg Psychiatry 2020 October; 91 (10): 1085-1091). NJD target this critical region and block the nerve impulse normally transmitted to a muscle to facilitate movement or its contraction. Myasthenic syndromes (myasthenia gravis and Eaton-Lambert syndrome) affect the efficacy of synaptic transmission through either immunological or genetic processes. In these pathologies, the absolute number of NMJs remain roughly the same but their efficacy to trigger a muscle action potential in response to motor neuron stimulation is decreased leading to fatigable muscle weakness (Gilhus N E et al Curr Opin Neurol. 2012 October; 25 (5): 523-9). In addition, it is recognized that designing treatments to strengthen and stabilize the remaining NMJs, the common final pathway of diseases with LMN degeneration, would be equally beneficial for ALS, spinal muscular atrophy (SMA) and spinal-bulbar muscular atrophy (SBMA) patients.

Only one treatment is currently approved by the United States Food and Drug Administration (FDA) and the European Medicines Agency (EMA) for ALS patients. Riluzole is an oral drug that blocks glutamatergic neurotransmission in the CNS. It is thought these effects may be partly due to inactivation of voltage-dependent sodium channels on glutamatergic nerve terminals. Riluzole also blocks some of the postsynaptic effects of glutamic acid by non-competitive blockade of N-methyl-D-aspartate (NMDA) receptors. Still, Riluzole has no effect on motor and respiratory functions and is neither suited for advanced forms of ALS nor for other motor neuron diseases.

The free radical scavenger Edaravone injected intravenously showed efficacy in a small subset of people with ALS in a phase 3 clinical trial. The study showed a significantly smaller decline of ALS Functional Rating Scale-Revised score compared with placebo. Up to now there is no indication that Edaravone might be effective in a wider population of patients with ALS who do not meet the criteria (Abe et al., Lancet Neurol. 2017, 16 (7), 505-512) and the application for a marketing authorization from the EMA for ALS treatment was withdrawn.

No specific treatment is available for neuromuscular junction disorders besides corticosteroids or immunosuppressants which could have serious side effects.

N-(3-(4-(3-(diisobutylamino)propyl)piperazin-1-yl)propyl)-1H-benzo[d]imidazol-2-amine sulphate salts and solvates thereof, previously disclosed in WO 2014/102339 are useful for the treatment and/or prevention of neurodegenerative diseases, amyloidopathies, tauopathies and developmental disease. These compounds are especially of interest for Alzheimer's disease, Parkinson's disease and tauopathies: a phase 2A clinical trial with N-(3-(4-(3-(diisobutylamino)propyl)piperazin-1-yl)propyl)-1H-benzo[d]imidazol-2-amine di-sulphate salt for patients suffering from progressive supranuclear palsy (PSP) is ongoing.

These salts are able to modulate Tau phosphorylation and increase progranulin (PGRN) neurotrophic factor levels which deficiency is known to accelerates Tau deposition and phosphorylation, as evidenced in human tau-expressing mice (J. Neuropathol. Exp. Neurol. 74, 158-165 (2015)). Therefore, these compounds are especially useful for the treatment of tauopathies such as fronto-temporal dementia and were thus proposed for treating ALS-FTD specifically.

Limited treatments are available to patients suffering from motor neuron diseases or neuromuscular junction disorders and there is still a need in the art for new chemical entities that could be used in the treatment or prophylaxis of these diseases, hence the Applicant investigated the potential of using N-(3-(4-(3-(diisobutylamino)propyl)piperazin-1-yl)propyl)-1H-benzo[d]imidazol-2-amine succinate salts and solvates thereof for such purpose.

### SUMMARY OF THE INVENTION

The present invention is based on the unexpected findings that succinate salts of N-(3-(4-(3-(diisobutylamino)propyl)piperazin-1-yl)propyl)-1H-benzo[d]imidazol-2-amine have neuroprotective effects on motor neurons and neuromuscular junctions.

The invention thus concerns N-(3-(4-(3-(diisobutylamino)propyl)piperazin-1-yl)propyl)-1H-benzo[d]imidazol-2-amine succinate salts and solvates thereof for use in the treatment and/or prevention of motor neuron diseases and neuromuscular junction disorders.

Moreover, the Applicant has shown that N-(3-(4-(3-(diisobutylamino)propyl)piperazin-1-yl)propyl)-1H-benzo[d]imidazol-2-amine succinate salts and solvates thereof were able to reduce the abnormal translocation of TDP-43 from the nucleus to the cytoplasm in motor neurons, a pathological feature observed in most ALS cases.

### DETAILED DESCRIPTION OF THE INVENTION

Applicant has shown that N-(3-(4-(3-(diisobutylamino)propyl)piperazin-1-yl)propyl)-1H-benzo[d]imidazol-2-amine succinate salts and pharmaceutically acceptable solvates thereof exert beneficial effects on primary culture of both wild-type rat and SOD1^{G93A} tg rat spinal cord motor neurons, by promoting their survival and the integrity of their neurite network after glutamate-induced damages. Furthermore, abnormal translocation of TDP-43 from the nucleus to the cytoplasm of motor neurons is a pathological feature observed in most ALS cases. The results presented in the examples section show that said succinate salts could also reverse the abnormal translocation of TDP-43 from the nucleus to the cytoplasm under glutamatergic stress.

Moreover, N-(3-(4-(3-(diisobutylamino)propyl)piperazin-1-yl)propyl)-1H-benzo[d]imidazol-2-amine succinate salts and pharmaceutically acceptable solvates thereof successfully protected the neuromuscular junctions and the neurite network against the glutamatergic stress in a co-culture of myoblasts and spinal cord explants.

All these findings support that N-(3-(4-(3-(diisobutylamino)propyl)piperazin-1-yl)propyl)-1H-benzo[d]imidazol-2-amine succinate salts and pharmaceutically acceptable solvates thereof could be an effective therapy to improve motor neuron diseases such as ALS, SMA and SBMA as well as neuromuscular junction disorders like myasthenia gravis and Eaton-Lambert syndrome.

Motor neuron diseases and neuromuscular junction disorders include, but are not limited to non-FTD amyotrophic lateral sclerosis, primary lateral sclerosis (PLS), hereditary spastic paraplegia (HSP), neurolathyrism, Konzo, Tay-Sachs disease, Sandhoff disease, progressive muscular atrophy (PMA), monomelic amyotrophy, spinal muscular atrophy (SMA), spinal-bulbar muscular atrophy (SBMA), progressive bulbar palsy (PBP), post-poliomyelitis syndrome, post-irradiation syndrome, stiff-person syndrome, disorders of the motor units resulting from an accident, myasthenia gravis and Eaton-Lambert syndrome.

Non-FTD amyotrophic lateral sclerosis designates the forms of sporadic or familial amyotrophic lateral sclerosis which are not associated with frontotemporal dementia, and includes amyotrophic lateral sclerosis with parkinsonism-dementia complex 1 (ALS-PDC), familial amyotrophic lateral sclerosis caused by mutation of one of the genes encoding superoxide dismutase 1 (SOD1), TAR DNA binding protein of 43-kDa (TDP-43), RNA-binding protein (FUS/TLS: Fused in Sarcoma/Translocated in Sarcoma) or ubiquilin 2 (UBQLN2). In ALS, patients with bulbar onset progress more rapidly than patients with limb onset or with a LMN presentation. Recent descriptions of regional variants suggest some patients have ALS isolated to a single spinal region, including brachial amyotrophic diplegia, leg amyotrophic diplegia, and isolated bulbar palsy, all of which herein being variants of non-FTD amyotrophic lateral sclerosis. Non-FTD ALS also includes respiratory-onset ALS, a rare variant that accounts for about 3% of all cases of ALS in which the initial symptoms are difficulty breathing with exertion at rest.

Primary lateral sclerosis (PLS) is a type of motor neuron disease that causes nerves within the brain to slowly break down. This makes the nerves unable to activate the motor neurons in the spinal cord, which control muscles.

Hereditary spastic paraplegia (HSP), also known as familial spastic paraparesis (FSP), is caused by upper motor neurons that slowly degenerate causing progressive spasticity and weakness of the legs. This leads to difficulty walking. As degeneration continues, symptoms worsen including impaired vision, ataxia, epilepsy, cognitive impairment, peripheral neuropathy, and/or deafness.

Neurolathyrism is caused by a toxin coming from the consumption of large quantities of certain legumes of the genus Lathyrus containing high concentrations of the glutamate analogue neurotoxin β-oxalyl-L-α,β-diaminopropionic acid (ODAP). ODAP is a poison of mitochondria, leading to excess cell death, especially in motor neurons; this toxin causes paralysis, characterized by lack of strength in or inability to move the lower limbs, and may involve pyramidal tracts producing signs of upper motor neuron damage.

Progressive muscular atrophy (PMA) also known as Duchenne-Aran muscular atrophy is clinically characterized by signs of lower motor neuron dysfunction and may evolve into ALS. Symptoms of PMA include atrophy, muscle weakness, lack of reflexes and lack of spasticity, symptoms can be limited to the arms, legs, or both.

Monomelic amyotrophy, also known as benign focal amyotrophy, juvenile segmental atrophy and Hirayama disease, is a rare benign lower motor neuron disorder characterized by muscular weakness and wasting in the distal upper extremities during adolescence followed by a spontaneous halt in progression and a stabilization of symptoms.

Spinal muscular atrophy (SMA) is a disease that deprives people of physical strength by affecting the motor nerve cells in the spinal cord, taking away the ability to walk, eat, or breathe. SMA is caused by a mutation in the survival motor neuron gene 1 (SMN1). that is critical to the function of the nerves that control muscles. Without it, nerve cells cannot properly function and eventually die, leading to debilitating and sometimes fatal muscle weakness.

Spinal-bulbar muscular atrophy (SBMA), also known as bulbospinal muscular atrophy and Kennedy disease, is a genetic disorder in which loss of motor neurons affects voluntary muscle movement, in particular the facial and swallowing muscles, and the arm and leg muscles, particularly those nearest the center of the body.

Progressive bulbar palsy (PBP) involves both upper and lower motor. This form of MND often causes difficulties with speech or swallowing. If the lower motor neurones are affected, the tongue tends to atrophy with visible fasciculation and reduced mobility. This results in a rather nasal type of speech. If the upper motor neurons are affected, the tongue is spastic and tends to cause dysarthria, difficulty with the mechanics of speech.

Stiff-person syndrome, also known as stiff-person spectrum disorder, is a neurological disorder affecting the brain and spinal cord causing fluctuating trunk and limb stiffness, painful muscle spasms, task-specific phobia, an exaggerated startle response, and ankylosing deformities such as fixed lumbar hyperlordosis.

Disorders of the motor units resulting from an accident” as used herein, refers to disorders wherein the motor units have been impaired due to a nerve lesion following an accident, a domestic or a traffic accident for instance.

Myasthenia gravis is an autoimmune disorder that impairs acetylcholine transmission at the neuromuscular junction, i.e. antibodies that attack the acetylcholine receptors resulting in muscle weakness. The most common symptoms of myasthenia gravis are weak, drooping eyelids, Weak eye muscles which cause double vision and excessive weakness of affected muscles after they are used.

Eaton-Lambert syndrome is an autoimmune disease where antibodies interfere with the release of the neurotransmitter acetylcholine receptors at the neuromuscular junction. It causes muscle weakness that tends to begin in the hip and thigh muscles, then typically spreads to the shoulder muscles, and then down the arms and legs to the hands and feet. The nerves that connect the head, face, eyes, nose, muscles, and ears to the brain (cranial nerves) are affected last.

In one embodiment, the succinate salts of N-(3-(4-(3-(diisobutylamino)propyl)piperazin-1-yl)propyl)-1H-benzo[d]imidazol-2-amine are those of Formula I

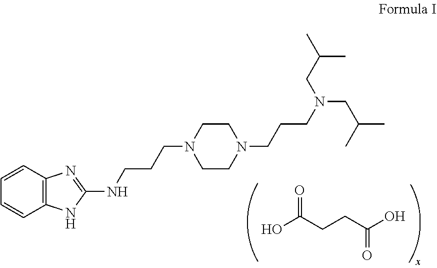

wherein x is 1 to 4, preferably x is 1.4 to 3.1, more preferably x is 1.4 to 1.6, still more preferably x is about 1.5, even more preferably x is 1.5.

In other words, the succinate salt of N-(3-(4-(3-(diisobutylamino)propyl)piperazin-1-yl)propyl)-1H-benzo[d]imidazol-2-amine contains 1 to 4 equivalents, preferably 1.4 to 3.1 equivalents, more preferably 1.4 to 1.6 equivalents, still more preferably about 1.5 equivalents and even more preferably 1.5 equivalents of succinate for one molecule of N-(3-(4-(3-(diisobutylamino)propyl)piperazin-1-yl)propyl)-1H-benzo[d]imidazol-2-amine.

In one preferred embodiment, the succinate salt of Formula I is the sesqui-succinate salt of N-(3-(4-(3-(diisobutylamino)propyl)piperazin-1-yl)propyl)-1H-benzo[d]imidazol-2-amine.

In one embodiment, preferred succinate salts are the compounds of Formula I wherein x is 2.9 to 3.1, preferably wherein x is about 3, more preferably x is 3.

In another preferred embodiment, the succinate salt of Formula I is the tris-succinate salt of N-(3-(4-(3-(diisobutylamino)propyl)piperazin-1-yl)propyl)-1H-benzo[d]imidazol-2-amine.

In a particular embodiment, the succinate salt of Formula I is in the form of a pharmaceutically acceptable solvate, preferably a hydrate. The solvate stoichiometry is between 0.4 to 2, preferably between 0.4 to 1.2, more preferably between 0.5 to 1.1, still more preferably about 0.5 or about 1.1, even more preferably 0.5 or 1.1 molecules of solvate for 1 molecule of succinate salt of Formula I.

In one embodiment, the succinate salt of Formula I is N-(3-(4-(3-(diisobutylamino)propyl)piperazin-1-yl)propyl)-1H-benzo[d]imidazol-2-amine sesqui-succinate salt hemi-hydrate.

In another embodiment, the succinate salt of Formula I is N-(3-(4-(3-(diisobutylamino)propyl)piperazin-1-yl)propyl)-1H-benzo[d]imidazol-2-amine sesqui-succinate salt mono-hydrate.

In one embodiment, one particularly preferred succinate salt is the compound of Formula I wherein x is 1.5 under the crystalline form having an X-ray powder diffraction (XRPD) pattern comprising peaks at diffraction angles of 2θ=3.8°±0.2°, 10.3°±0.2°, 12.4°±0.2°, 16.2°±0.2°, 17.9°±0.2°, 19.8°±0.2°, 20.4°±0.2°, 23.8°±0.2° and 26.7°±0.2° when irradiated with a CuKα light source. This specific crystalline form is referred to as “Form 1” throughout the application and its XRPD diffractogram recorded on a PANalytical X'pert pro with PIXcel detector (128 channels) is shown in FIG. 1.

Another preferred succinate salt is the compound of Formula I wherein x is 3 under the crystalline form having an X-ray powder diffraction (XRPD) pattern comprising peaks at diffraction angles of 2θ=5.3°±0.2°, 15.0°±0.2°, 15.3°±0.2°, 16.8°±0.2°, 17.9°±0.2°, 20.5°±0.2°, 21.2°±0.2°, 23.9°±0.2°, 24.3°±0.2° and 26.6°±0.2° when irradiated with a CuKα light source. This specific crystalline form is referred to as “Form 2” throughout the application and its XRPD diffractogram recorded on a PANalytical X'pert pro with PIXcel detector (128 channels) is shown in FIG. 2.

Another preferred succinate salt is hemi-hydrate of the compound of Formula I wherein x is 1.5, and under the crystalline form having an X-ray powder diffraction (XRPD) pattern comprising peaks at diffraction angles of 2θ=3.8°±0.2°, 4.4°±0.2°, 9.2°±0.2°, 10.6°±0.2°, 13.8°±0.2°, 17.7°±0.2°, 19.9°±0.2°, 21.6°±0.2° and 23.6°±0.2° when irradiated with a CuKα light source. Its XRPD diffractogram recorded on a PANalytical X'pert pro with PIXcel detector (128 channels) is shown in FIG. 3.

Another preferred succinate salt is the mono-hydrate of the compound of Formula I wherein x is 1.5, and under the crystalline form having an X-ray powder diffraction (XRPD) pattern comprising peaks at diffraction angles of 2θ=4.2±0.2°, 4.4°±0.2°, 9.6°±0.2°, 10.6°±0.2°, 15.0°±0.2°, 16.3°±0.2°, 17.4°±0.2°, 19.7°±0.2°, 22.8°±0.2°, 29.1 and 29.7°±0.2° when irradiated with a CuKα light source. Its XRPD diffractogram recorded on a PANalytical X'pert pro with PIXcel detector (128 channels) is shown in FIG. 4.

N-(3-(4-(3-(diisobutylamino)propyl)piperazin-1-yl)propyl)-1H-benzo[d]imidazol-2-amine succinate salts and pharmaceutically acceptable solvates are thus useful as a medicament, in particular for treating or preventing motor neuron diseases, neuromuscular junction disorders and all diseases wherein abnormal translocation of TDP-43 from the nucleus to the cytoplasm in motor neurons is observed.

Hence, the invention also concerns a succinate salt of N-(3-(4-(3-(diisobutylamino)propyl)piperazin-1-yl)propyl)-1H-benzo[d]imidazol-2-amine or a pharmaceutically acceptable solvate thereof as defined herein for use in treating and/or preventing motor neuron diseases and neuromuscular junction disorders, in particular selected from non-FTD amyotrophic lateral sclerosis, primary lateral sclerosis (PLS), hereditary spastic paraplegia (HSP), neurolathyrism, Konzo, Tay-Sachs disease, Sandhoff disease, progressive muscular atrophy (PMA), monomelic amyotrophy, spinal muscular atrophy (SMA), spinal-bulbar muscular atrophy (SBMA), progressive bulbar palsy (PBP), post-poliomyelitis syndrome, post-irradiation syndrome, stiff-person syndrome, disorders of the motor units resulting from an accident, myasthenia gravis, Eaton-Lambert syndrome. Preferably, the disease is selected from non-FTD amyotrophic lateral sclerosis, primary lateral sclerosis (PLS), hereditary spastic paraplegia (HSP), progressive muscular atrophy (PMA), monomelic amyotrophy, spinal muscular atrophy (SMA), spinal-bulbar muscular atrophy (SBMA), progressive bulbar palsy (PBP), myasthenia gravis, Eaton-Lambert syndrome disorders of the motor units resulting from an accident. More preferably, the disease is selected from non-FTD amyotrophic lateral sclerosis, primary lateral sclerosis (PLS), hereditary spastic paraplegia (HSP), spinal muscular atrophy (SMA), spinal-bulbar muscular atrophy (SBMA), progressive bulbar palsy (PBP). Even more preferably, the disease is non-FTD amyotrophic lateral sclerosis.

In other terms, the invention also provides for a method of treating and/or preventing a motor neuron disease or a neuromuscular junction disorder, in particular those cited above as well as embodiments thereof, comprising administering to a patient in need thereof a pharmaceutically effective amount of a N-(3-(4-(3-(diisobutylamino)propyl)piperazin-1-yl)propyl)-1H-benzo[d]imidazol-2-amine succinate salt or a pharmaceutically acceptable solvate thereof as described herein. In a particular embodiment, the disease is selected from non-FTD amyotrophic lateral sclerosis, primary lateral sclerosis (PLS), hereditary spastic paraplegia (HSP), spinal muscular atrophy (SMA), spinal-bulbar muscular atrophy (SBMA), progressive bulbar palsy (PBP).

In other terms, the invention also provides for the use of N-(3-(4-(3-(diisobutylamino)propyl)piperazin-1-yl)propyl)-1H-benzo[d]imidazol-2-amine succinate salt or a pharmaceutically acceptable solvate thereof as described herein, in the manufacture of a medicament for treating and/or preventing a motor neuron disease or a neuromuscular junction disorder, in particular those cited above as well as embodiments thereof. In a particular embodiment, the disease is selected from non-FTD amyotrophic lateral sclerosis, primary lateral sclerosis (PLS), hereditary spastic paraplegia (HSP), spinal muscular atrophy (SMA), spinal-bulbar muscular atrophy (SBMA), progressive bulbar palsy (PBP).

In one particular embodiment, the invention also concerns N-(3-(4-(3-(diisobutylamino)propyl)piperazin-1-yl)propyl)-1H-benzo[d]imidazol-2-amine succinate salt or a pharmaceutically acceptable solvate thereof as defined herein for use in delaying in a patient the onset of motor neuron diseases and neuromuscular junction disorders, in particular selected from non-FTD amyotrophic lateral sclerosis, primary lateral sclerosis (PLS), hereditary spastic paraplegia (HSP), neurolathyrism, Konzo, Tay-Sachs disease, Sandhoff disease, progressive muscular atrophy (PMA), monomelic amyotrophy, spinal muscular atrophy (SMA), spinal-bulbar muscular atrophy (SBMA), progressive bulbar palsy (PBP), post-poliomyelitis syndrome, post-irradiation syndrome, stiff-person syndrome, disorders of the motor units resulting from an accident, myasthenia gravis, Eaton-Lambert syndrome. Preferably, the disease is selected from non-FTD amyotrophic lateral sclerosis, primary lateral sclerosis (PLS), hereditary spastic paraplegia (HSP), progressive muscular atrophy (PMA), monomelic amyotrophy, spinal muscular atrophy (SMA), spinal-bulbar muscular atrophy (SBMA), progressive bulbar palsy (PBP), myasthenia gravis, Eaton-Lambert syndrome, disorders of the motor units resulting from an accident. More preferably, the disease is selected from non-FTD amyotrophic lateral sclerosis, primary lateral sclerosis (PLS), hereditary spastic paraplegia (HSP), spinal muscular atrophy (SMA), spinal-bulbar muscular atrophy (SBMA), progressive bulbar palsy (PBP). Even more preferably, the disease is non-FTD amyotrophic lateral sclerosis.

In other terms, the invention provides for a method for delaying in a patient the onset of motor neuron diseases or neuromuscular junction disorders, in particular those cited above as well as embodiments thereof, comprising administering to a patient in need thereof a pharmaceutically effective amount of a N-(3-(4-(3-(diisobutylamino)propyl)piperazin-1-yl)propyl)-1H-benzo[d]imidazol-2-amine succinate salt or a pharmaceutically acceptable solvate thereof. In a particular embodiment, the disease is selected from non-FTD amyotrophic lateral sclerosis, primary lateral sclerosis (PLS), hereditary spastic paraplegia (HSP), spinal muscular atrophy (SMA), spinal-bulbar muscular atrophy (SBMA), progressive bulbar palsy (PBP).

In other terms, the invention also provides for the use of N-(3-(4-(3-(diisobutylamino)propyl)piperazin-1-yl)propyl)-1H-benzo[d]imidazol-2-amine succinate salt or a pharmaceutically acceptable solvate thereof as described herein, in the manufacture of a medicament for delaying in a patient the onset of a motor neuron disease or a neuromuscular junction disorder, in particular those cited above as well as embodiments thereof. In a particular embodiment, the disease is selected from non-FTD amyotrophic lateral sclerosis, primary lateral sclerosis (PLS), hereditary spastic paraplegia (HSP), spinal muscular atrophy (SMA), spinal-bulbar muscular atrophy (SBMA), progressive bulbar palsy (PBP).

According to a further feature of the present invention there is provided a method for reducing abnormal translocation of TDP43 from the nucleus to the cytoplasm in motor neurons, in a patient, preferably a warm blooded animal, and even more preferably a human, in need of such treatment, which comprises administering to said patient an effective amount of N-(3-(4-(3-(diisobutylamino)propyl)piperazin-1-yl)propyl)-1H-benzo[d]imidazol-2-amine succinate salt or a pharmaceutically acceptable solvate thereof.

According to one embodiment, the succinate salts of N-(3-(4-(3-(diisobutylamino)propyl)piperazin-1-yl)propyl)-1H-benzo[d]imidazol-2-amine, as well as their pharmaceutical acceptable solvates may be administered as part of a combination therapy. Thus, are included within the scope of the present invention embodiments comprising co-administration of compositions and medicaments which contain, in addition to a succinate salt of N-(3-(4-(3-(diisobutylamino)propyl)piperazin-1-yl)propyl)-1H-benzo[d]imidazol-2-amine or a pharmaceutically acceptable solvate thereof as active ingredient, additional therapeutic agents and/or active ingredients. Such multiple drug regimens, often referred to as “combination therapy”, may be used in the treatment and/or prevention of any motor neuron disease or neuromuscular junction disorder. The use of such combinations of therapeutic agents is especially pertinent with respect to the treatment of the above-mentioned motor neuron diseases within a patient in need of treatment or one at risk of becoming such a patient.

In addition to the requirement of therapeutic efficacy, which may necessitate the use of active agents in addition to the succinate salts of N-(3-(4-(3-(diisobutylamino)propyl)piperazin-1-yl)propyl)-1H-benzo[d]imidazol-2-amine or pharmaceutically acceptable solvates thereof, there may be additional rationales which compel or highly recommend the use of combinations of drugs involving active ingredients which represent adjunct therapy, i.e., which complement and supplement the function performed by the succinate salts of N-(3-(4-(3-(diisobutylamino)propyl)piperazin-1-yl)propyl)-1H-benzo[d]imidazol-2-amine or pharmaceutically acceptable solvates thereof. Suitable supplementary therapeutic agents used for the purpose of auxiliary treatment include drugs which, instead of directly treating and/or preventing a disease or condition mediated by or associated with motor neuron degeneration, neuromuscular junction degeneration and/or pathological accumulation of TDP-43 in the cytoplasm of motor neurons, treat diseases or conditions which directly result from or indirectly accompany said degenerations.

According to a further feature of the present invention, a succinate salt of N-(3-(4-(3-(diisobutylamino)propyl)piperazin-1-yl)propyl)-1H-benzo[d]imidazol-2-amine, a pharmaceutically acceptable solvate thereof may be used in combination therapy with other drugs used for treating motor neuron diseases such as ALS, SMA, SBMA and neuromuscular junction disorders such as myasthenia gravis and Eaton-Lambert syndrome. More particularly, the compound of Formula I, as well as pharmaceutically acceptable solvates thereof, may be used as an adjunct therapy in combination with riluzole, edaravone, pyridostigmine, inhibitors of glucosylceramide degradation such as ambroxol and conduritol B epoxide, acetylcholine release inducers such as guanidine, corticosteroids such as prednisolone, antiseizure drugs such as carbamazepine and phenytoin, or drugs that are currently under clinical trial for the treatment of ALS as disclosed in Van Eijk et al., Current opinion in neurology 2020, 33 (5), 655.

Thus, the methods of treatment and pharmaceutical compositions of the present invention may employ a succinate salt of N-(3-(4-(3-(diisobutylamino)propyl)piperazin-1-yl)propyl)-1H-benzo[d]imidazol-2-amine or a pharmaceutically acceptable solvate thereof in monotherapy of motor neuron diseases and neuromuscular junction disorders. However, said methods and compositions may also be used multiple therapy in which one or more N-(3-(4-(3-(diisobutylamino)propyl)piperazin-1-yl)propyl)-1H-benzo[d]imidazol-2-amine succinate salts or their pharmaceutically acceptable solvates are co-administered in combination with one or more other therapeutic agents.

In the above-described embodiment, combinations of a N-(3-(4-(3-(diisobutylamino)propyl)piperazin-1-yl)propyl)-1H-benzo[d]imidazol-2-amine succinate salt or a pharmaceutically acceptable solvate thereof and other therapeutic active agents may be administered, in terms of dosage forms, either separately or in conjunction with each other, and in terms of their time of administration, either serially or simultaneously. Thus, the administration of one component agent may be prior to, concurrent with, or subsequent to the administration of the other component agent(s).

Generally, for pharmaceutical use, the succinate salts of N-(3-(4-(3-(diisobutylamino)propyl)piperazin-1-yl)propyl)-1H-benzo[d]imidazol-2-amine or pharmaceutically acceptable solvates thereof may be formulated as a pharmaceutical composition comprising least at one salt succinate of N-(3-(4-(3-(diisobutylamino)propyl)piperazin-1-yl)propyl)-1H-benzo[d]imidazol-2-amine or a pharmaceutically acceptable solvate thereof and at least one pharmaceutically acceptable carrier, diluent, excipient and/or adjuvant, and optionally one or more further therapeutic agents and/or active ingredients.

By means of non-limiting examples, pharmaceutical composition may be in a dosage form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences. The pharmaceutical compositions may be formulated in solid form and re-dissolved or suspended prior to use. Preferred pharmaceutical compositions of the succinate salts of the invention or solvates thereof are in a solid dosage form suitable for oral administration.

Some preferred, but non-limiting examples of dosage forms include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, agar, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propyl-hydroxybenzoates, talc, magnesium stearate, edible oils, vegetable oils and mineral oils or suitable mixtures thereof. Particularly preferred dosage forms of the succinate salts of the invention and solvates thereof are soft and hard capsules, in particular soft and hard gelatin capsules, preferably hard gelatin capsules, which are formulated with at least one microcrystalline cellulose excipient, especially Avicel® PH101. The pharmaceutical compositions can optionally contain other substances that are commonly used in pharmaceutical formulations, such as lubricating agents, wetting agents, emulsifying and suspending agents, dispersing agents, disintegrating agents, stabilizing agents, isotonic agents, bulking agents, fillers, preserving agents, sweetening agents, flavouring agents, perfuming agents, colouring agents, antibacterial agents and/or antifungal agents such as parabens, chlorobutanol, phenol, sorbic acid, dispensing agents, flow regulators, release agents, etc. The compositions may also be formulated so as to provide rapid, sustained or delayed release of the active compound(s) contained therein.

The pharmaceutical compositions of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 0.05 and 1000 mg, and usually between 1 and 500 mg, of the at least one compound of the invention, e.g. about 10, 25, 50, 100, 200, 300 or 400 mg per unit dosage.

Usually, depending on the condition to be prevented or treated and the route of administration, the active compound of the invention will usually be administered between 0.01 to 100 mg per kilogram, more often between 0.1 and 50 mg, such as between 1 and 25 mg, for example about 0.5, 1, 2, 5, 10, 15, 20 or 25 mg, per kilogram body weight of the patient per day, which may be administered as a single daily dose, divided over one or more daily doses, or essentially continuously, e.g. using a drip infusion.

All references to compounds of the invention and in particular compounds of Formula I include references to solvates, in particular hydrates, multi-component complexes and liquid crystals thereof.

The compounds disclosed throughout the present application were named using ChemDraw® Ultra version 11.0 (CambridgeSoft, Cambridge, MA, USA).

N-(3-(4-(3-(diisobutylamino)propyl)piperazin-1-yl)propyl)-1H-benzo[d]imidazol-2-amine free base can be obtained as disclosed in WO 2006/051489. The succinate salts and solvates thereof can be prepared according to techniques known in the art such as those involving precipitation, crystallization, recrystallization, lyophilisation, phase transfer or ion exchange resins.

### Definitions

The definitions and explanations below are for the terms as used throughout the entire application, including both the specification, the figures and the claims.

Unless otherwise stated any reference to compounds of the invention herein, comprises the compound of Formula I as such as well as its pharmaceutically acceptable solvates. All references to compounds of Formula I include references to pharmaceutically acceptable solvates thereof and all crystalline forms thereof.

The term “administration”, or a variant thereof (e.g., “administering”), means providing the active agent or active ingredient (e.g. N-(3-(4-(3-(diisobutylamino)propyl)piperazin-1-yl)propyl)-1H-benzo[d]imidazol-2-amine), alone or as part of a pharmaceutically acceptable composition, to the patient in whom/which the condition, symptom, or disease is to be treated or prevented.

The term “human” refers to a subject of both genders and at any stage of development (i.e. neonate, infant, juvenile, adolescent, adult).

The term “patient” refers to a warm-blooded animal, more preferably a human, who/which is awaiting the receipt of, or is receiving medical care or is/will be the object of a medical procedure.

By “pharmaceutically acceptable” is meant that the ingredients of a pharmaceutical composition are compatible with each other and not deleterious to the patient thereof.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The expression “reducing” as used herein refers to a partial reduction or a complete reduction.

The term “sesqui” as used herein, refers to one and a half equivalent, a sesqui-succinate salt of a compound means its succinic acid addition salt wherein the succinic acid is present in a ratio of 3:2 compared with the compound in its free base form.

The term “solvate” is used herein to describe a compound in this invention that contains stoichiometric or sub-stoichiometric amounts of one or more pharmaceutically acceptable solvent molecule such as ethanol. The term “hydrate” is employed when said solvent is water. The pharmaceutically acceptable solvent molecules may be co-crystallized with the compound of the invention, and/or be present in crystalline and/or amorphous phases of solids thereof, and/or be adsorbed thereto.

The term “therapeutically effective amount” (or more simply an “effective amount”) as used herein means the amount of active agent or active ingredient (e.g. N-(3-(4-(3-(diisobutylamino)propyl)piperazin-1-yl)propyl)-1H-benzo[d]imidazol-2-amine) that is sufficient to achieve the desired therapeutic or prophylactic effect in the patient to which/whom it is administered.

The terms “treat”, “treating” and “treatment, as used herein, are meant to include alleviating, attenuating or abrogating a condition or disease and/or its attendant symptoms.

The present invention will be better understood with reference to the following examples. These examples are intended to representative of specific embodiments of the invention, and are not intended as limiting the scope of the invention.

### BRIEF DESCRIPTION OF THE DRAWINGS

FIG. 1 shows the XRPD 2Θ diffractogram of Form 1, crystalline form of N-(3-(4-(3-(diisobutylamino)propyl)piperazin-1-yl)propyl)-1H-benzo[d]imidazol-2-amine sesqui-succinate salt.

FIG. 2 shows the XRPD 2Θ diffractogram of Form 2, crystalline form of N-(3-(4-(3-(diisobutylamino)propyl)piperazin-1-yl)propyl)-1H-benzo[d]imidazol-2-amine tris-succinate salt.

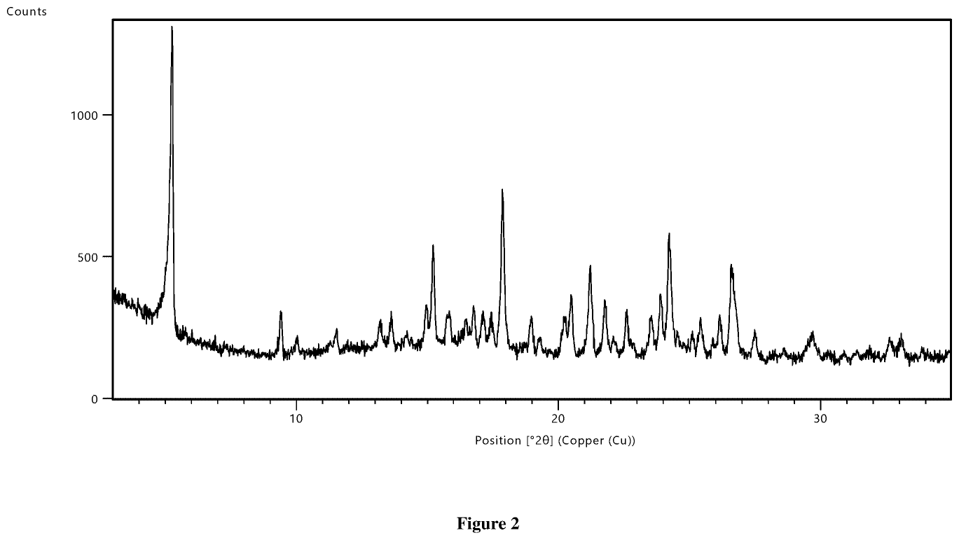

FIG. 3 shows the XRPD 2Θ diffractogram of a crystalline form of N-(3-(4-(3-(diisobutylamino)propyl)piperazin-1-yl)propyl)-1H-benzo[d]imidazol-2-amine sequi-succinate salt hemi-hydrate (0.5 eq. of water).

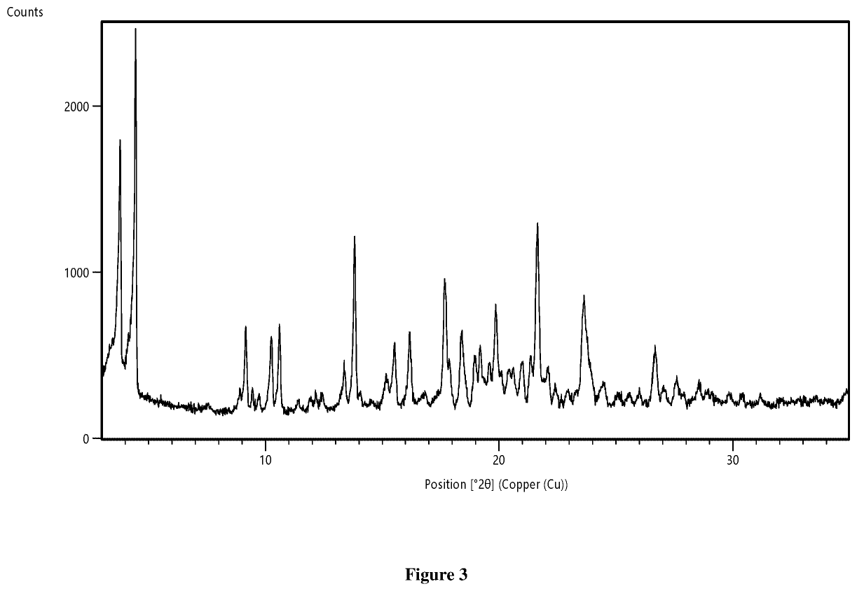

FIG. 4 shows the XRPD 2Θ diffractogram of a crystalline form of N-(3-(4-(3-(diisobutylamino)propyl)piperazin-1-yl)propyl)-1H-benzo[d]imidazol-2-amine sesqui-succinate salt mono-hydrate (1.1 eq. of water).

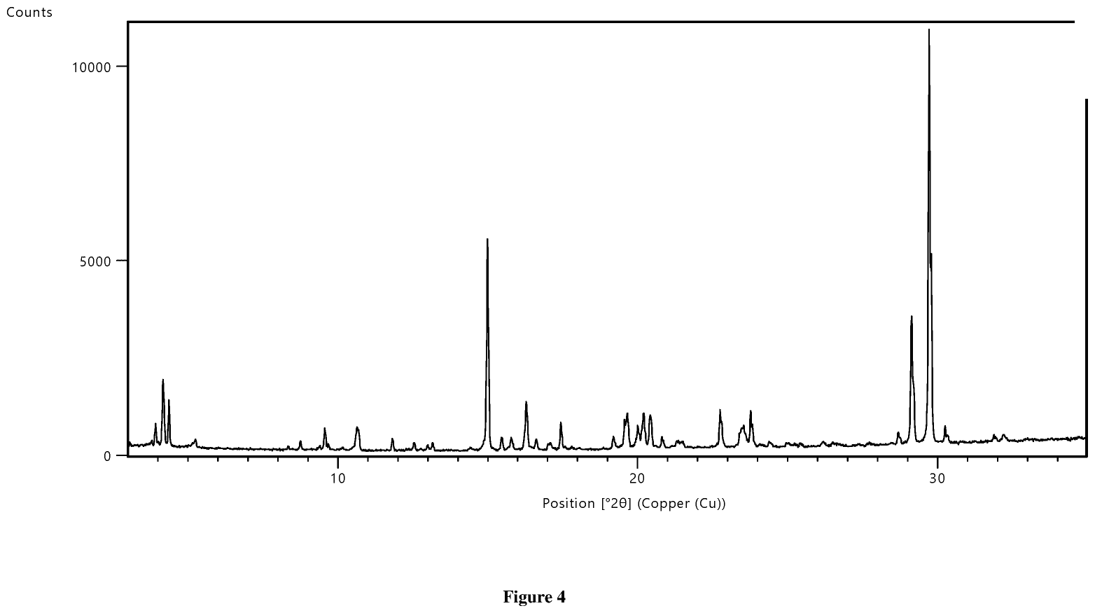

FIG. 5 shows the ¹H-NMR spectrum of Form 1, crystalline form of N-(3-(4-(3-(diisobutylamino)propyl)piperazin-1-yl)propyl)-1H-benzo[d]imidazol-2-amine sesqui-succinate salt (DMSO-d₆, 500.12 MHz).

FIG. 6 shows the ¹³C Quant-NMR Spectrum of Form 1, crystalline form of N-(3-(4-(3-(diisobutylamino)propyl)piperazin-1-yl)propyl)-1H-benzo[d]imidazol-2-amine sesqui-succinate salt (DMSO-d₆, 500.12 MHz).

FIG. 7 shows the effect of an incubation of N-(3-(4-(3-(diisobutylamino)propyl)piperazin-1-yl)propyl)-1H-benzo[d]imidazol-2-amine) sesqui-succinate salt Form 1 for 48 h after 20 min of glutamate injury, in a primary culture of rat spinal motor neurons on survival (A), neurite network (B) and on extra-nuclear TDP-43 (eTDP43) (C) of MAP-2 positive MNs. Results are expressed as a percentage of control as mean±SEM (n=4-6/group). One-way ANOVA followed by PLSD Fisher's test.

FIG. 8 shows the effect of a 24h pre-incubation of N-(3-(4-(3-(diisobutylamino)propyl)piperazin-1-yl)propyl)-1H-benzo[d]imidazol-2-amine) sesqui-succinate salt Form 1 before 20 min of glutamate injury plus 24h post-injury incubation, in a primary culture of rat spinal motor neurons on survival (A), neurite network (B) and on extra-nuclear TDP-43 (eTDP43) (C) of MAP-2 positive MNs. Results are expressed as a percentage of control as mean±SEM (n=4-6/group). One-way ANOVA followed by PLSD Fisher's test.

FIG. 9 shows the effects of an incubation of N-(3-(4-(3-(diisobutylamino)propyl)piperazin-1-yl)propyl)-1H-benzo[d]imidazol-2-amine) sesqui-succinate salt Form 1 for 48h after 20 min of glutamate injury, in comparison to Riluzole (Ril) and Edaravone (Eda), in a primary culture of SOD1^{G93A} Tg rat spinal cord motor neurons on survival (A), neurite network (B) and on extranuclear TDP43 (eTDP43) of MAP-2 positive MNs (C). Results are expressed as a percentage of control as mean+/−SEM (n=5-6). One-way ANOVA followed by PLSD Fisher's test.

FIG. 10 shows the effect of N-(3-(4-(3-(diisobutylamino)propyl)piperazin-1-yl)propyl)-1H-benzo[d]imidazol-2-amine) sesqui-succinate salt Form 1, in comparison to Riluzone (Ril) in a coculture of spinal cord explant and myoblasts injured with glutamate: (A) number of NMJs, (B) size of NMJs, and (C) neurite network of NFH (+) MN. Results are expressed as a percentage of control as mean+/−SEM (n=5-6). One-way ANOVA followed by PLSD Fisher's test.

### CHEMISTRY EXAMPLES

The following abbreviations are used throughout the present application: ° C.: Celsius degrees, DMSO: dimethylsulfoxyde, δ: NMR chemical shifts expressed in ppm, eq: equivalent(s), EDTA: ethylenediamine tetra-acetic acid, eq.: equivalent(s), g: gram(s), h: hour(s), HPLC: high performance liquid chromatography, Hz: Hertz, L: liter(s), M: mol/L, mM: mmol/L, μM: μmol/L, Me: methyl, MEK: methyl ethyl ketone, mg: milligram(s), MHz: megahertz, min: minute(s), mL: milliliter(s), mm: millimeter(s), mol: mole(s), mmol: millimole(s), μmol: micromole(s), nm: nanometer(s), NMR: nuclear magnetic resonance, PBS: phosphate-buffered saline, ppm: party per million, RH: relative humidity, RPM: round(s) per minute, rt: retention time, RT: room temperature (ca 15-25° C.), s: second(s), TFA: trifluoroacetic acid, UV: ultraviolet, v: volume, XRPD: X-ray powder diffraction.

All reported temperatures are expressed in degrees Celsius (° C.); all reactions were carried out at room temperature (RT) unless otherwise stated.

Experimental set-up or purification procedures that were used in this invention, when not described in specific details, are assumed to be known to those conversant in the art and are described in such standard reference manuals such as: i) Gordon, A. J.; Ford, R. A. “The Chemist's Companion—A Handbook of Practical Data, Techniques, and References”, Wiley: New York, 1972; ii) Vogel's Textbook of Practical Organic Chemistry, Pearson Prentice Hall: London, 1989; iii) P. Heinrich Stahl and Camille G. Wernuth ‘Handbook of Pharmaceutical Salts’, Wiley VCH.

HPLC analysis.

Instrument: Dionex Ultimate 3000

Column: Supelco Ascentis Express C18 15×4.6 mm 2.7 μm

Column Temperature: 50° C.

Autosampler Temperature: Ambient

UV wavelength: 275 nm

Injection Volume: 20 μL

Flow Rate: 1 mL/min

Mobile Phase A: 0.1% TFA in Water

Mobile Phase B: 0.1% TFA in Acetonitrile

Gradient program:

| Time (minutes) | Solvent B [%] |
|---|---|
| 0.0 | 5 |
| 30.0 | 60 |
| 33.0 | 60 |
| 33.1 | 5 |
| 40.0 | 5 |

### NMR Analysis

NMR experiments were performed on a Bruker A VIIIHD spectrometer equipped with a DCH cryoprobe operating at 500.12 MHz for protons and carbons. Experiments were performed in deuterated DMSO and each sample was prepared to ca. 10 mM concentration. Further analysis was optionally performed in D₂O and/or methanol-d₄. Chemical shifts are expressed in parts per million, (ppm, δ units). Coupling constants are expressed in Hz. Abbreviations for multiplicities observed in NMR spectra are as follows: s (singlet), d (doublet), t (triplet), q (quadruplet), m (multiplet), br (broad).

### X-Ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Mylar polymer film to support the sample. The multi-well plate was then placed into the diffractometer and analysed using Cu Kα radiation (α₁ λ=1.54060 Å; α₂=1.54443 Å; β=1.39225 Å; α₁:α2 ratio=0.5) running in transmission mode (step size 0.0130° 2θ, step time 18.87s) using 40 kV/40 mA generator settings. Data were visualized and images generated using the HighScore Plus 4.7 desktop application (PANalytical, 2017).

Solvents and reagents were purchased and used as received from commercial vendors unless otherwise specified.

### Example 1: Synthesis and Characterisation of Form 1: Crystalline Form of N-(3-(4-(3-(diisobutylamino)propyl)piperazin-1-yl)propyl)-1H-benzo[d]imidazol-2-amine Sesqui-Succinate Salt

514.6 mg of N-(3-(4-(3-(diisobutylamino)propyl)piperazin-1-yl)propyl)-1H-benzo[d]imidazol-2-amine was weighed out in a 20 mL scintillation vial. 10 mL of methyl ethyl ketone (99% pure) was added to the vial which was stirred at 40° C. using a temperature-controlled block, a stirrer plate, and magnetic needle. No clear solution was observed so further 5 mL of MEK was added to aid dissolution at 40° C. A clear solution was obtained; the clear solution at 40° C. was polish-filtered using PTFE filter (0.45 microns) and a syringe. The filtered solution was added back to a clean 20 mL scintillation vial and re-heated to 40° C. 142.5 mg of succinic acid (1 mole equivalent) was added to the vial. A clear solution was initially observed. After stirring at 40° C. for 30 minutes, crystallization of material was observed. The experiment was temperature cycled between 40° C. and 5° C. in 3-hour cycles for 20 hours. At 5° C., the slurry was filtered using Buchner flask and funnel using grade 1 Whatman filter paper (pore size=11 μm) under vacuum. The wet cake was washed with 2.5 mL of chilled (5° C.) MEK and dried under reduced pressure at 40° C. for 2 hours. The dried isolated solid was analysed by XRPD and HPLC. The filtered mother liquor was analysed for concentration by HPLC.

An isolated yield of 78.5% was achieved, with an HPLC yield of 93.5% determined based on the mother liquor concentration (see Table 1).

TABLE 1
Results from Form 1 succinate salt preparation

| | Input Free | Yields (%) | | Solid purity | Losses to mL's | Losses to wash |
|---|---|---|---|---|---|---|
| Solvent system | Base (mg) | Theoretical | Isolated | % Area | (mg/mL) | (mg/mL) |
| Methyl ethyl | 514 | 93.5 | 78.5 | 99.2 | 11.1 | 5.5 |
| ketone | | | | | | |

HPLC indicated a purity of 99.2%; rt=10.65 min.

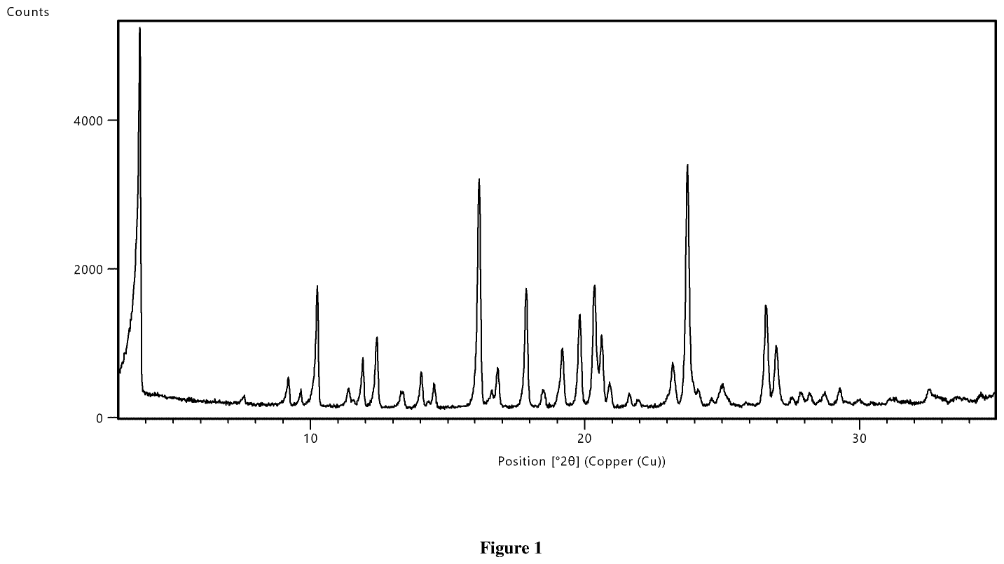

The XRPD 2Θ diffractogram showed the material to be crystalline (FIG. 1) and comprised peaks at diffraction angles of 2θ=3.8°±0.2° (100%), 10.3°±0.2° (11.3%), 12.4°±0.2° (6.8%), 16.2°±0.2° (23.5%), 17.9°±0.2° (11.9%), 19.8°±0.2° (10.2%), 20.4°±0.2° (13.0%), 23.8°±0.2° (23.3%) and 26.7°±0.2° (9.8%) when irradiated with a CuKα light source.

Another batch of N-(3-(4-(3-(diisobutylamino)propyl)piperazin-1-yl)propyl)-1H-benzo[d]imidazol-2-amine, sesqui-succinate salt was made and further characterized by HPLC, XRPD and NMR.

HPLC indicated a purity of 98.4%.

XRPD 2Θ diffractogram was identical to that of previously described batch.

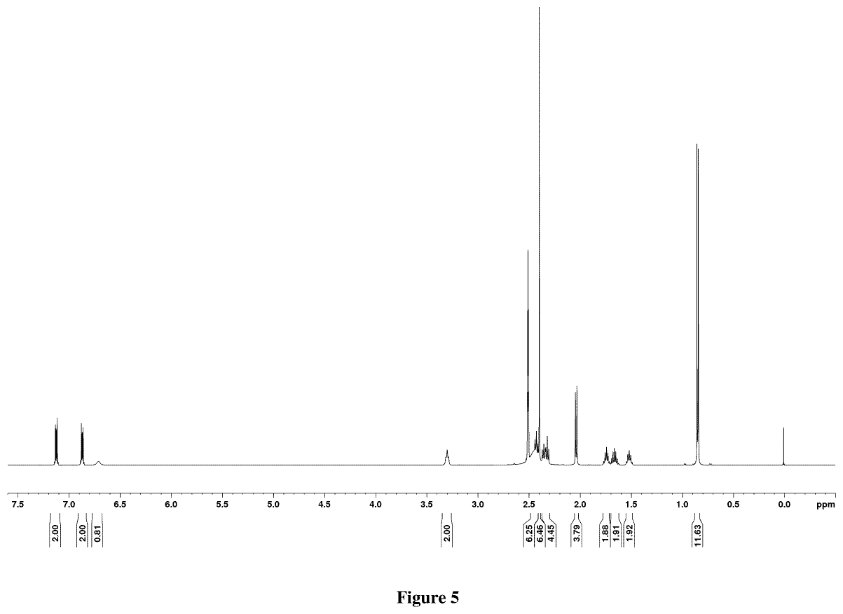

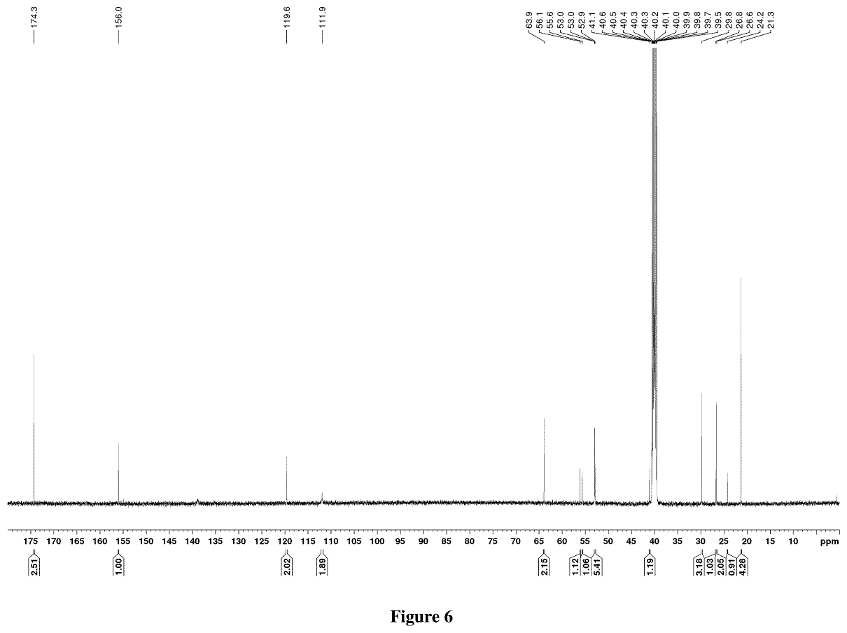

¹H and quantitative ¹³C NMR spectra in DMSO-d₆ are presented in FIG. 5 and FIG. 6 respectively.

¹H NMR spectrum (500.12 MHz-DMSO-d₆): δ (ppm): 7.13 (2H dd J1=6 Hz, J2=3 Hz), 6.87 (2H dd J1=6 Hz, J2=3 Hz), 6.71 (1H br s), 3.30 (2H t J=7 Hz), 2.42 (6H m, 2.33 4H dt J1=16 Hz, J2-7 Hz), 2.04 (4H d J=7 Hz), 1.74 (2H quintet J=7 Hz), 1.67 (2H septet J=7 Hz), 1.52 (2H quintet J=7 Hz), 0.85 (12H d J=7 Hz) ppm.

Quantitative ¹³C NMR spectrum (121.16 MHz-DMSO-d₆): δ (ppm): 174.3, 156.0, 119.5, 111.9, 63.9 56.1, 55.6, 53.0 (d), 29.8, 26.8, 26.6, 24.2, 21.3. Quantitative 13C NMR confirmed the stoichiometry and successful formation of a sesqui-succinate salt.

### BIOLOGY EXAMPLES

### Example 1: effect of N-(3-(4-(3-(diisobutylamino)propyl)piperazin-1-yl)propyl)-1H-benzo[d]imidazol-2-amine) Sesqui-Succinate Form 1 on Rat Primary Motor Neurons Injured with Glutamate

### Primary Culture of Spinal Cord Motor Neurons

Rat spinal cord motor neurons (MN) were cultured as previously described by Boussicault et al., J Neurosci Res. 2020 December; 98 (12), 2435-50 and Wang et al. Hum. Mol. Genet., 2013-12-1; 22 (23), 4706-19. Pregnant female rats of 14 days gestation (Rats Wistar; Janvier Labs France) were killed using a deep anesthesia with CO₂ chamber followed by cervical dislocation. Then, embryos (E14) were removed from the uterus and immediately placed in ice-cold L15 Leibovitz medium with a 2% penicillin (10,000 U/mL) and streptomycin (10 mg/mL) solution (PS) and 1% bovine serum albumin (BSA). Spinal cords were removed and placed in ice-cold medium of Leibovitz (L15). Spinal cords were treated for 20 min at 37° C. with a trypsin-Ethylenediaminetetraacetic acid (EDTA) solution at a final concentration of 0.05% trypsin and 0.02% EDTA. The dissociation was stopped by the addition of Dulbecco's modified Eagle's medium (DMEM) with 4.5 g/liter of glucose, containing DNAse I grade II (final concentration 0.5 mg/mL) and 10% fetal calf serum (FCS). Cells were mechanically dissociated by three forced passages through the tip of a 10-mL pipette. Cells were then centrifuged at 515×g for 10 min at +4° C. The supernatant was discarded, and the pellet was resuspended in a defined culture medium consisting of Neurobasal medium with a 2% solution of B27 supplement, 2 mmol/liter of L-glutamine, 2% of PS solution, and 10 ng/ml of brain-derived neurotrophic factor (BDNF). Viable cells were counted in a Neubauer cytometer, using the trypan blue exclusion test. The cells were seeded at a density of 20,000 per well in 96-well plates precoated with poly-L-lysine and cultured at 37° C. in an air (95%)-CO₂ (5%) incubator. The medium was changed every 2 days.

### Compound Treatment—48h Incubation

On day 13 of culture, glutamate was added to a final concentration of 5 μM diluted in control medium in presence of the sesqui-succinate Form 1 for 20 min. After 20 min, glutamate was washed and fresh culture medium with the sesqui-succinate Form 1 was added for 48 hours.

### End Point Evaluation

After 15 days of culture (48h after the glutamate injury), the cell culture supernatant was collected and the spinal cord MNs were fixed by a cold solution of ethanol (95%) and acetic acid (5%) for 5 min at −20° C. for immunostaining. Cells were washed twice in PBS. Cell membranes were permeabilized and non-specific sites were blocked with a solution of PBS containing 0.1% of saponin and 1% FCS for 15 min at room temperature. Then, cells were incubated for 2 hours with:
a) mouse monoclonal antibody anti-microtubule associated protein 2 (MAP-2) at dilution of 1/400 in PBS containing 1% fetal calf serum and 0.1% of saponin. This antibody binds specifically MAP-2 present in cell bodies and neurites of all MNs. This antibody was revealed with Alexa Fluor 488 goat anti-mouse IgG at the dilution 1/400 in PBS containing 1% FCS, 0.1% saponin, for 1 hour at room temperature.
b) Rabbit polyclonal antibody anti-TDP43 at dilution of 1/100 in PBS containing 1% fetal calf serum and 0.1% of saponin. Cytoplasmic localization of TDP43 was investigated. This antibody was revealed with Alexa Fluor 568 goat anti-rabbit IgG at a dilution of 1/400 in PBS containing 1% FCS, 0.1% saponin, for 1 hour at room temperature.

Nuclei of neurons were counterstained with the fluorescent dye (Hoechst).

For each condition, 30 pictures (representative of the whole well area) per well were automatically taken using ImageXpress (Molecular Devices) with 20× magnification. All images were taken under the same conditions (exposition time, gain and laser intensity). Analysis of different endpoints, were automatically performed using Custom Module Editor (Molecular Devices). Motoneurons (MAP-2) were distinguished from interneurons (staining MAP-2) using the following morphological criteria: a cell body diameter >15 mm and the presence of a minimum of three neuritic processes (Ferraiuolo et al. Brain 2011:134; 2627-2641).

The endpoints were:
survival of MNs (MAP-2 staining number of MNs)
neurite network of MNs (MAP-2 staining, total neurite length expressed in μm)
cytoplasmic TDP43 (extranuclear, eTDP43) in MAP-2 positive MNs (overlapping between MAP-2 and cytoplasmic TDP-43 in μm²). Results are expressed as the ratio of the overlapping MAP-2/TDP-43 per number of MNs.

### Statistical Analysis

All values are expressed as mean+/−SEM. Statistical analyses were performed using one-way ANOVA followed by PLSD Fisher test. All tests were performed using GraphPad Prism software version 9 and statistical significance was set at *p<0.05, **p<0.001, ***p<0.001 and ****p<0,0001.

### Results

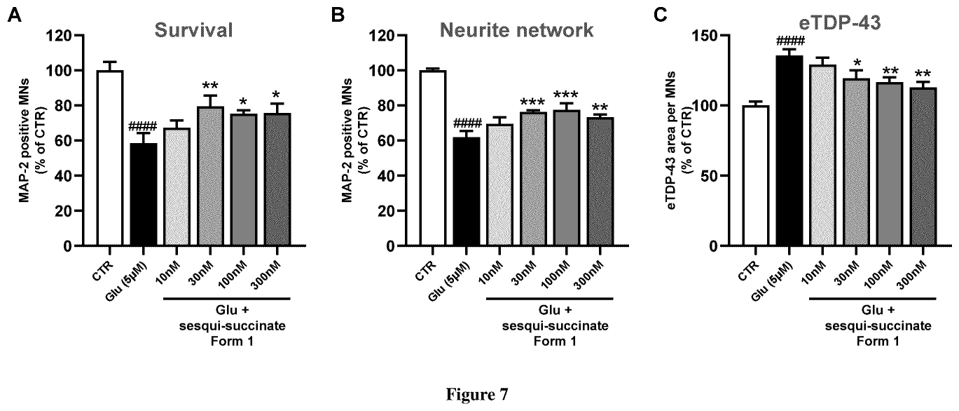

Motor neuron survival: as expected, the glutamate intoxication significantly decreased cell survival, as compared to the control group (mean: 58%; FIG. 7A). Several doses of N-(3-(4-(3-(diisobutylamino)propyl)piperazin-1-yl)propyl)-1H-benzo[d]imidazol-2-amine) sesqui-succinate Form 1 (30 nM to 300 nM) showed a positive and significant effect on survival, as compared to the glutamate condition. The maximal effect was obtained with the dose at 30 nM (mean: 79%).
Neurite network integrity: glutamate significantly reduced the neurite network (mean: 62%, FIG. 7B). Several doses of N-(3-(4-(3-(diisobutylamino)propyl)piperazin-1-yl)propyl)-1H-benzo[d]imidazol-2-amine) sesqui-succinate Form 1 (30 nM to 300 nM) application were able to improve the neurite network after glutamate injury, with a maximal effect at 100 nM (mean: 77%).
Extranuclear TDP-43: glutamate application significantly increased abnormal cytoplasmic TDP-43 signal (mean: 135% compared to control; FIG. 7C). The sesqui-succinate Form 1 at concentrations of 30 to 300 nM was able to significantly reduce the accumulation of TDP-43 in the cytoplasm, up to 17% at the 300 nM concentration.

### Example 2: Effect of N-(3-(4-(3-(diisobutylamino)propyl)piperazin-1-yl)propyl)-1H-benzo[d]imidazol-2-amine) sesqui-succinate Form 1 pre-treatment on rat primary motor neurons injured with glutamate 24h pre-treatment-injury-24h treatment

### Primary Culture of Spinal Cord Motor Neurons

The culture was carried out as described in example 1.

### Compound Treatment −24h Pre-Incubation +24h Incubation

On day 12 of culture, primary motor neurons were treated with the sesqui-succinate Form 1 for 24 hours. On day 13 of culture, glutamate was added to a final concentration of 5 UM diluted in control medium in presence of the sesqui-succinate salt Form 1 for 20 min. After 20 min, glutamate was washed and fresh culture medium with the sesqui-succinate Form 1 was added for an additional 24 hours.

### End Point Evaluation

The end point evaluation was carried out as described in example 1.

### Statistical Analysis

The statistical analysis was carried out as described in example 1.

### Results

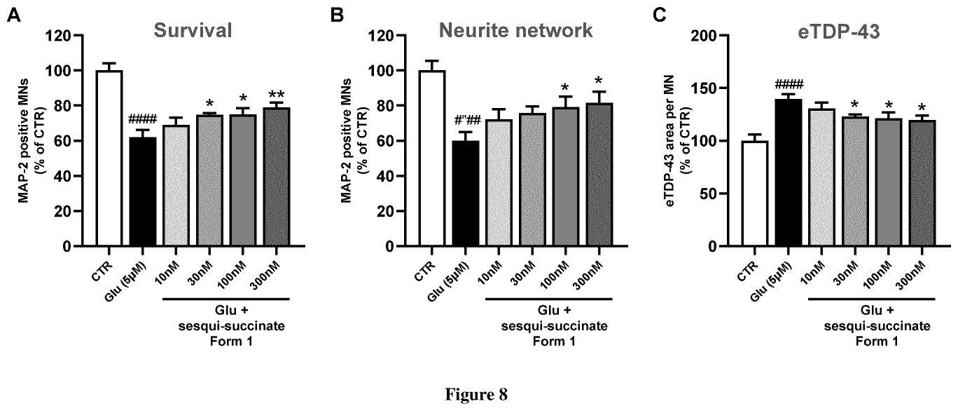

Motor neuron survival: as expected, glutamate significantly decreased cell survival, as compared to the control group (mean: 62%; FIG. 8A). Several doses of the sesqui-succinate salt Form 1 (30 nM to 300 nM) displayed positive and significant effect on survival, as compared to the glutamate condition. The maximal effect was obtained with the dose of sesqui-succinate salt Form 1 at 300 nM (mean survival: 79%).
Neurite network integrity: glutamate strongly impaired the neurite network of spinal motor neurons (mean: 60%; FIG. 8B). All doses from 30 nM to 300 nM of the sesqui-succinate salt Form 1 were able to protect the neurite network from glutamate damage, with a maximal effect at 300 nM (mean: 81%).
Extranuclear TDP-43: glutamate application significantly increased cytoplasmic TDP43 signal (mean: 139% compared to control, FIG. 8C). The sesqui-succinate salt Form 1 (at the doses of 30 nM to 300 nM) was also able to reduce the abnormal distribution of TDP-43 by up to 14%.

### Example 3: Effect of N-(3-(4-(3-(diisobutylamino)propyl)piperazin-1-yl)propyl)-1H-benzo[d]imidazol-2-amine) Sesqui-Succinate Salt Form 1 on Rat Primary SOD1 Motor Neuron Maturation after a Glutamate Injury

Genetic rodent models have been used to study ALS pathogenesis, including rats over-expressing human superoxide dismutase 1 (SOD1) with mutations known to cause human familial ALS (eg. SOD1^{G93A} rats). The ALS rat model expressing the mutated form of hSOD-1^{G93A} exhibits features that closely recapitulate the clinical and histopathologic features of the human disease (Nagai et al., The Journal of Neuroscience, Dec. 1, 2001, 21 (23): 9246-9254). In human or rodent studies (SOD1 models), MN loss is preceded by increased excitability. As increased neuronal excitability correlates with structural changes in dendritic arbors and spines, dendritic atrophy and spine loss in striatal medium spiny neurons (MSNs) and lower MNs of lumbar spinal cord are observed (Ferrucci et al., Neurobiology of Disease 37 (2010) 370-383; Avossa et al., Neuroscience 138 (2006) 1179-1194).

### Genotyping of Embryos for SOD1^{G93A} Motoneurons

Female (Sprague Dawley, Janvier) were mated at Neuro-Sys (Gardanne, France) with Tg SOD1^{G93A} male rats (Taconic Bioscience, US). Pregnant females of 14 days of gestation were used for the culture. The day of the dissection, a piece of each embryo head (˜3 mm³) was placed in a 2 mL DNase free tube with a new scalpel. The DNA was extracted with the SYBR Green Extract-N-Amp tissue PCR kit (Sigma Aldrich). Briefly, 120 μL of extraction solution was put on each piece of embryo heads. Then, they were incubated for 10 min at room temperature. At the end of this incubation period, the heads were incubated for 5 min at 95° C. Immediately after this last incubation, 100 μL of neutralizing solution was added; each DNA extract was diluted at 1/40 and stored at +4° C. until use. SOD1^{G93A} gene was determined using genomic fragment with human SOD1 primers (5′-CATCAGCCCTAATCCATCTGA-3′ SEQ ID NO: 1; 5′-CGCGACTAACAATCAAAGTGA-3′ SEQ ID NO:2). The SOD1 primers were diluted at 3 μM in sterile ultrapure water. Briefly, a mix for PCR was prepared with ultrapure water (4 μL per sample), primer at 3 μM (2 μL per sample) and Master Mix (10 μL per sample). In a PCR 96 wells plate, 16 μL of PCR mix was added in each well. 4 μL of each diluted DNA was added according to a plan deposit.

The RT-PCR was run using the CFX96 Biorad RT-PCR system, using the following program:
Initial denaturation: 95° C.-20 sec
45 cycles: 95° C.-10 sec, 65° C.-10 sec, 72° C.-30 sec
Melt curve: 95° C.-15 sec, 64° C.-1 min, 90° C.-30 sec, 60° C.-15 sec

The amplification plots and melt curves were analyzed thanks to the Biorad software.

The results for each sample were compared to negative control (ultrapure water) and to the positive control (DNA from Tg SOD1^{G93A} embryos), to conclude on the genotype of each embryo (WT or Tg).

### Primary Culture of Spinal Cord SOD1^{G93A} Motor Neurons

Rat spinal cord motor neurons were cultured as described by Boussicault et al., J Neurosci Res. 2020 December; 98 (12), 2435-50 and Wang et al. Hum. Mol. Genet., 2013 December 1; 22 (23), 4706-19. Pregnant female rats of 14 days of gestation were killed by cervical dislocation. The culture was carried out as described in example 1.

### Compound Treatment −48h Incubation

On day 13, culture medium was removed and cultures were exposed to the test compounds: N-(3-(4-(3-(diisobutylamino)propyl)piperazin-1-yl)propyl)-1H-benzo[d]imidazol-2-amine) sesqui-succinate salt Form 1, in comparison to Riluzole and Edaravone, and glutamate (5 μM) for 20 min at 37° C. in defined medium. After glutamate exposure, the cultures were washed with defined medium at 37° C. then placed in fresh culture medium containing the test compound, Riluzole or Edavarone for additional 48h.

### End Point Evaluation

The end point evaluation was carried out as described in example 1.

### Statistical Analysis

The statistical analysis was carried out as described in example 1.

### Results

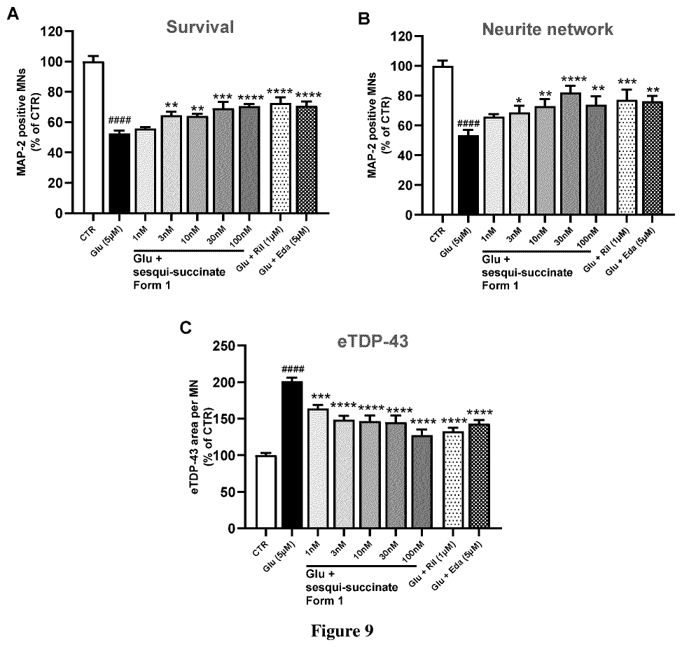

The injury with glutamate significantly reduced the number of MNs, the length of their neurite network and triggered an abnormal distribution of TDP-43 towards the cytoplasm (see FIG. 9).

The 3 nM to 100 nM doses of N-(3-(4-(3-(diisobutylamino)propyl)piperazin-1-yl)propyl)-1H-benzo[d]imidazol-2-amine) sesqui-succinate salt Form 1 were neuroprotective. The protective effects on neuronal survival and on the distribution of TDP-43 were dose-dependent. At a dose of 100 nM, the protective effects of N-(3-(4-(3-(diisobutylamino)propyl)piperazin-1-yl)propyl)-1H-benzo[d]imidazol-2-amine) sesqui-succinate salt Form 1 were comparable to those of Riluzole or Edaravone.

### Example 4: Effect of N-(3-(4-(3-(diisobutylamino)propyl)piperazin-1-yl)propyl)-1H-benzo[d]imidazol-2-amine) Sesqui-Succinate Salt Form 1 on Spinal Cord/Muscle Neuromuscular Junctions Injured with Glutamate

### Culture of Human Myoblasts and Rat Spinal Cord Explants

All experiments were carried out in accordance with the National Institutes of Health Guide for the Care and Use of Laboratory Animals and followed current European Union regulations (Directive 2010/63/EU).

The human muscle cell line was established from dissociated cells (22 000 cells per wells). They were plated in gelatin-coated 0.1% in water on 48 wells plate and were allowed to grow in a proliferation medium consisting of mix of 62% of MEM medium and 25% of M199 medium supplemented with glutamine 2 mM, human insulin 10 μg/mL, human recombinant epidermal growth factor 10 ng/mL (EGF), human recombinant fibroblast growth factor basic 2 ng/mL (bFGF), fetal calf scrum 10% (FCS) and 2% of penicillin 10.000 U/mL and streptomycin 10.000 μg/mL (PS). The medium was changed every 2 days.

Five days after the start of culture, immediately after the fusion of satellite cells, whole transverse slices of spinal cords with 4 dorsal root ganglia (DRG) attached, taken from 13-day-old rat Wistar embryos (Janvier Labs, France), were placed on the muscle monolayer (one explant per well in the central area). The presence of DRG is necessary to achieve a good ratio of innervation. Innervated cultures were maintained in a mixed (67%/25%) medium composed of MEM and medium 199, supplemented with 5% FCS, insulin 5 μg/mL, glutamine 2 mM and 2% PS. After 24h of co-culture, neurite elongation from spinal cord explants is usually observed. These neurites made contacts with myotubes and induced the formation of neuromuscular junctions and the first contractions were observed after ˜8 days of co-culture. Quickly thereafter, innervated muscle fibers located in the proximity to the spinal cord explants, were virtually continuously contracting. Innervated fibers were morphologically and spatially distinct from the non-innervated ones and could easily be distinguished from them. The plates were maintained at 37° C. in a humidified incubator, in an atmosphere of air (95%)-CO₂ (5%).

### Compound Treatment −48h Incubation

On day 27 (co-culture), co-cultures were incubated with N-(3-(4-(3-(diisobutylamino)propyl)piperazin-1-yl)propyl)-1H-benzo[d]imidazol-2-amine) sesqui-succinate salt Form 1 or Riluzole in the presence of and glutamate (final concentration of 60 μM diluted in control medium), for 20 min.

After 20 min of glutamate intoxication, the supernatant was removed and fresh culture medium with N-(3-(4-(3-(diisobutylamino)propyl)piperazin-1-yl)propyl)-1H-benzo[d]imidazol-2-amine) sesqui-succinate salt Form 1 or with Riluzole was added for an additional 48h.

### End Point Evaluation

### Immunostaining

After 48 hours of intoxication, cells were incubated with 500 nM a-bungarotoxin coupled with Alexa 488 during 15 min in the culture medium at 37° C. to detect motor endplates. After 2 washing in PBS, cells were fixed by a solution of 4% of paraformaldehyde in PBS, pH=7.3 for 20 min at room temperature.

The cells were washed twice in PBS. A solution of PBS containing 0.1% of saponin and 1% FCS for 15 min at room temperature to permeabilized cells and block non-specific sites.

Then, co-cultures were incubated with a mouse monoclonal anti-neurofilament 200 KD antibody (NFH) at the dilution of 1/400 in PBS containing 1% FCS, 0.1% saponin, for 2 h at room temperature. Antibody against NFH stained neurites and the axon of motor neuron. This antibody was revealed with Alexa Fluor 568 goat anti-mouse IgG at the dilution 1/400 in PBS containing 1% FCS, 0.1% saponin, for 1 h at room temperature. Nuclei of neurons were labeled by Hoechst solution, a nuclear fluorescent marker at 1 μm/ml in the same solution.

Analysis of different endpoints were automatically performed. One co-culture was done (6 wells per conditions). For each condition, 20 pictures per well were automatically taken using ImageXpress (Molecular devices) with 10× magnification. All the images were taken under the same conditions.

The following endpoints were automatically measured:
(1) number of NMJs,
(2) mean size of NMJs (NMJ area in μm²),
(3) total neurite length (μm) (=innervation network).

### Statistical Analysis

The statistical analysis was carried out as described in example 1.

### Results

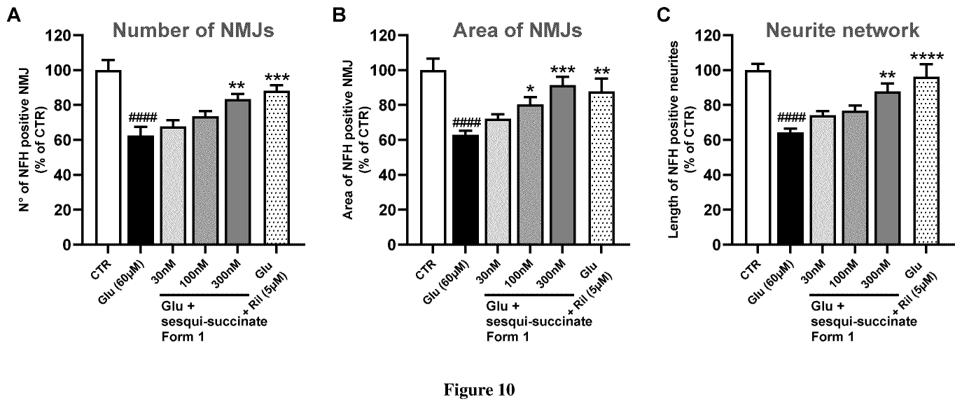

As shown in FIG. 10, the application of glutamate (60 M, 20 min) resulted in a significant reduction in the number (A) and the total area (B) of NMJs in the culture. The area of NMJs indicates the degree of clustering of their subunits. Functional NMJs are larger than not innervated NMJs. In addition, the injury led to a significant loss of MNs neurites (C). The sesqui-succinate salt Form 1 (300 nM) displayed neuroprotective effects on a dose-dependent manner, as it increased the number and the total area of NMJs and the total MNs neurite network. The protective effects of N-(3-(4-(3-(diisobutylamino)propyl)piperazin-1-yl)propyl)-1H-benzo[d]imidazol-2-amine) sesqui-succinate salt Form 1 were comparable to those of Riluzole.

1. A method for treating and/or preventing motor neuron diseases and neuromuscular junction disorders selected from non-FTD amyotrophic lateral sclerosis, primary lateral sclerosis (PLS), hereditary spastic paraplegia (HSP), neurolathyrism, Tay-Sachs disease, Sandhoff disease, progressive muscular atrophy (PMA), monomelic amyotrophy, spinal-bulbar muscular atrophy (SBMA), progressive bulbar palsy (PBP), post-irradiation syndrome, stiff-person syndrome, disorders of the motor units resulting from an accident, myasthenia gravis, Eaton-Lambert syndrome, comprising administering an effective amount of succinate salt of N-(3-(4-(3-(diisobutylamino)propyl)piperazin-1-yl)propyl)-1H-benzo[d]imidazol-2-amine and pharmaceutically acceptable solvates thereof.

2. A method for delaying in a patient the onset of motor neuron diseases and neuromuscular junction disorders selected from non-FTD amyotrophic lateral sclerosis, primary lateral sclerosis (PLS), hereditary spastic paraplegia (HSP), neurolathyrism, Tay-Sachs disease, Sandhoff disease, progressive muscular atrophy (PMA), monomelic amyotrophy, spinal-bulbar muscular atrophy (SBMA), progressive bulbar palsy (PBP), post-irradiation syndrome, stiff-person syndrome, disorders of the motor units resulting from an accident, myasthenia gravis, Eaton-Lambert syndrome, comprising administering an effective amount of succinate salt of N-(3-(4-(3-(diisobutylamino)propyl)piperazin-1-yl)propyl)-1H-benzo[d]imidazol-2-amine and pharmaceutically acceptable solvates thereof.

3. The method according to claim 1, wherein the succinate salt has Formula I 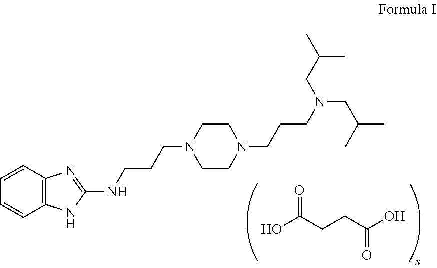
wherein x is 1 to 4, and pharmaceutically acceptable solvates thereof.

4. The method according to claim 3, wherein x is about 1.5.

5. The method according to claim 4, wherein the succinate salt is N-(3-(4-(3-(diisobutylamino)propyl)piperazin-1-yl)propyl)-1H-benzo[d]imidazol-2-amine sesqui-succinate.

6. The method according to claim 1, wherein the motor neuron diseases and neuromuscular junction disorders are selected from non-FTD amyotrophic lateral sclerosis, primary lateral sclerosis (PLS), hereditary spastic paraplegia (HSP), progressive muscular atrophy (PMA), monomelic amyotrophy, spinal-bulbar muscular atrophy (SBMA), progressive bulbar palsy (PBP), myasthenia gravis, Eaton-Lambert syndrome, disorders of the motor units resulting from an accident.

7. The method according to claim 6, wherein the motor neuron diseases are selected from non-FTD amyotrophic lateral sclerosis, primary lateral sclerosis (PLS), hereditary spastic paraplegia (HSP), spinal-bulbar muscular atrophy (SBMA), progressive bulbar palsy (PBP).

8. The method according to claim 7, wherein the motor neuron disease is non-FTD amyotrophic lateral sclerosis.

9. The method according to claim 4, wherein the succinate salt is N-(3-(4-(3-(diisobutylamino)propyl)piperazin-1-yl)propyl)-1H-benzo[d]imidazol-2-amine sesqui-succinate under the crystalline form having an X-ray powder diffraction (XRPD) pattern comprising peaks at diffraction angles of 2θ=3.8°±0.2°, 10.3°±0.2°, 12.4°±0.2°, 16.2°±0.2°, 17.9°±0.2°, 19.8°±0.2°, 20.4°±0.2°, 23.8°±0.2° and 26.7°±0.2° when irradiated with a CuKα light source.

10. The method according to claim 9, wherein the motor neuron diseases and neuromuscular junction disorders are selected from non-FTD amyotrophic lateral sclerosis, primary lateral sclerosis (PLS), hereditary spastic paraplegia (HSP), progressive muscular atrophy (PMA), monomelic amyotrophy, spinal-bulbar muscular atrophy (SBMA), progressive bulbar palsy (PBP), myasthenia gravis, Eaton-Lambert syndrome, disorders of the motor units resulting from an accident.

11. The method according to claim 10, wherein the motor neuron diseases are selected from non-FTD amyotrophic lateral sclerosis, primary lateral sclerosis (PLS), hereditary spastic paraplegia (HSP), spinal-bulbar muscular atrophy (SBMA), progressive bulbar palsy (PBP).

12. The method according to claim 11, wherein the motor neuron disease is non-FTD amyotrophic lateral sclerosis.

13. The method according to claim 2, wherein the succinate salt has Formula I 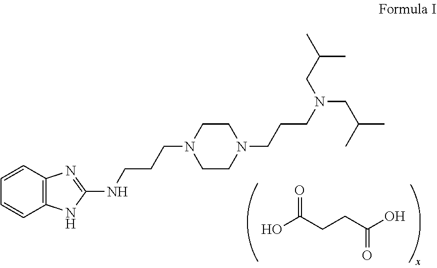
wherein x is 1 to 4, and pharmaceutically acceptable solvates thereof.

14. The method according to claim 13 and pharmaceutically acceptable solvates thereof, wherein x is about 1.5.

15. The method according to claim 14, wherein the succinate salt is N-(3-(4-(3-(diisobutylamino)propyl)piperazin-1-yl)propyl)-1H-benzo[d]imidazol-2-amine sesqui-succinate.

16. The method according to claim 15, wherein the succinate salt is N-(3-(4-(3-(diisobutylamino)propyl)piperazin-1-yl)propyl)-1H-benzo[d]imidazol-2-amine sesqui-succinate under the crystalline form having an X-ray powder diffraction (XRPD) pattern comprising peaks at diffraction angles of 2θ=3.8°±0.2°, 10.3°±0.2°, 12.4°±0.2°, 16.2°±0.2°, 17.9°±0.2°, 19.8°±0.2°, 20.4°±0.2°, 23.8°±0.2° and 26.7°±0.2° when irradiated with a CuKα light source.

17. The method according to claim 16, wherein the motor neuron diseases and neuromuscular junction disorders are selected from non-FTD amyotrophic lateral sclerosis, primary lateral sclerosis (PLS), hereditary spastic paraplegia (HSP), progressive muscular atrophy (PMA), monomelic amyotrophy, spinal-bulbar muscular atrophy (SBMA), progressive bulbar palsy (PBP), myasthenia gravis, Eaton-Lambert syndrome, disorders of the motor units resulting from an accident.

18. The method according to claim 17, wherein the motor neuron diseases are selected from non-FTD amyotrophic lateral sclerosis, primary lateral sclerosis (PLS), hereditary spastic paraplegia (HSP), spinal-bulbar muscular atrophy (SBMA), progressive bulbar palsy (PBP).

19. The method according to claim 18, wherein the motor neuron disease is non-FTD amyotrophic lateral sclerosis.


SEQUENCE LISTING (2 sequences)

SEQ ID NO: 1 - DNA, 21 bp, synthetic construct
  source 1..21: mol_type = other DNA
catcagccctaatccatctga

SEQ ID NO: 2 - DNA, 21 bp, synthetic construct
  source 1..21: mol_type = other DNA
cgcgactaacaatcaaagtga